SEQUENCE LISTING (48 sequences)

SEQ ID NO: 1 - protein, 18 aa, synthetic construct
DGKDYIALNEDLRSWTAA

SEQ ID NO: 2 - protein, 17 aa, synthetic construct
DLRSWTAADMAAQITKR

SEQ ID NO: 3 - protein, 17 aa, synthetic construct
EDLRSWTAADMAAQITK

SEQ ID NO: 4 - protein, 18 aa, synthetic construct
EDLRSWTAADMAAQITKR

SEQ ID NO: 5 - protein, 16 aa, synthetic construct
DLRSWTAADMAAQTTK

SEQ ID NO: 6 - protein, 17 aa, synthetic construct
DLRSWTAADMAAQTTKH

SEQ ID NO: 7 - protein, 17 aa, synthetic construct
EDLRSWTAADMAAQTTK

SEQ ID NO: 8 - protein, 18 aa, synthetic construct
EDLRSWTAADMAAQTTKH

SEQ ID NO: 9 - protein, 19 aa, synthetic construct
EDLRSWTAADMAAQTTKHK

SEQ ID NO: 10 - protein, 17 aa, synthetic construct
EDLSSWTAADTAAQITQ

SEQ ID NO: 11 - protein, 21 aa, synthetic construct
EDLSSWTAADTAAQITQRKWE

SEQ ID NO: 12 - protein, 20 aa, synthetic construct
LNEDLSSWTAADTAAQITQR

SEQ ID NO: 13 - protein, 23 aa, synthetic construct
DGKDYIALNEDLRSWTAADTAAQ

SEQ ID NO: 14 - protein, 14 aa, synthetic construct
EDLRSWTAADTAAQ

SEQ ID NO: 15 - protein, 16 aa, synthetic construct
EDLRSWTAADTAAQIT

SEQ ID NO: 16 - protein, 17 aa, synthetic construct
EDLRSWTAADTAAQITQ

SEQ ID NO: 17 - protein, 18 aa, synthetic construct
EDLRSWTAADTAAQITQR

SEQ ID NO: 18 - protein, 21 aa, synthetic construct
EDLRSWTAADTAAQITQRKWE

SEQ ID NO: 19 - protein, 16 aa, synthetic construct
LNEDLRSWTAADTAAQ

SEQ ID NO: 20 - protein, 15 aa, synthetic construct
NEDLRSWTAADTAAQ

SEQ ID NO: 21 - protein, 14 aa, synthetic construct
EDLRSWTAADTAAQ

SEQ ID NO: 22 - protein, 17 aa, synthetic construct
EDLRSWTAADTAAQITQ

SEQ ID NO: 23 - protein, 18 aa, synthetic construct
EDLRSWTAADTAAQITQR

SEQ ID NO: 24 - protein, 21 aa, synthetic construct
EDLRSWTAADTAAQITQRKWE

SEQ ID NO: 25 - protein, 16 aa, synthetic construct
LNEDLRSWTAADTAAQ

SEQ ID NO: 26 - protein, 19 aa, synthetic construct
LNEDLRSWTAADTAAQITQ

SEQ ID NO: 27 - protein, 14 aa, synthetic construct
EDLRSWTAVDTAAQ

SEQ ID NO: 28 - protein, 17 aa, synthetic construct
EDLRSWTAVDTAAQISE

SEQ ID NO: 29 - protein, 16 aa, synthetic construct
LNEDLRSWTAVDTAAQ

SEQ ID NO: 30 - protein, 13 aa, synthetic construct
DLRSWTAVDTAAQ

SEQ ID NO: 31 - protein, 12 aa, synthetic construct
LRSWTAVDTAAQ

SEQ ID NO: 32 - protein, 14 aa, synthetic construct
EDLRSWTAVDTAAQ

SEQ ID NO: 33 - protein, 16 aa, synthetic construct
DLRSWTAVDTAAQISE

SEQ ID NO: 34 - protein, 14 aa, synthetic construct
EDLRSWTAADTVAQ

SEQ ID NO: 35 - protein, 14 aa, synthetic construct
EDLRSWTAVDTAAQ

SEQ ID NO: 36 - protein, 16 aa, synthetic construct
DLRSWTAVDTAAQISE

SEQ ID NO: 37 - protein, 18 aa, synthetic construct
KPVTTGVSETVFLPREDH

SEQ ID NO: 38 - protein, 18 aa, synthetic construct
EDLRSWTAADMAAQITKR

SEQ ID NO: 39 - protein, 18 aa, synthetic construct
EDLRSWTAADMAAQTTKH

SEQ ID NO: 40 - protein, 18 aa, synthetic construct
EDLRSWTAADMAAQITQR

SEQ ID NO: 41 - protein, 21 aa, synthetic construct
EDLSSWTAADTAAQITQRKWE

SEQ ID NO: 42 - protein, 21 aa, synthetic construct
EDLSSWTAADTAAQITQLKWE

SEQ ID NO: 43 - protein, 21 aa, synthetic construct
EDLRSWTAADTAAQISQRKLE

SEQ ID NO: 44 - protein, 21 aa, synthetic construct
EDLRSWTAADTAAQITQRKWE

SEQ ID NO: 45 - protein, 21 aa, synthetic construct
EDLRSWTAADKAAQITQRKWE

SEQ ID NO: 46 - protein, 17 aa, synthetic construct
EDLRSWTAVDTAAQISE

SEQ ID NO: 47 - protein, 17 aa, synthetic construct
EDLRSWTAADTVAQITQ

SEQ ID NO: 48 - protein, 18 aa, synthetic construct
KPVTTGVSETVFLPREDH

(19) United States
(12) Patent Application Publication
(10) Pub. No.: US 2025/0216401 A1
(43) Pub. Date: Jul. 3, 2025

(54) METHOD OF DETECTING HUMAN LEUKOCYTE ANTIGENS (HLA)

(71) Applicant: THE TERASAKI INNOVATION CENTER, Glendale, CA (US)

(72) Inventors: Neng Jen Remi Shih, Winnetka, CA (US); Thoa Nong, Granada Hills, CA (US); Jar-How Lee, Los Angeles, CA (US)

(21) Appl. No.: 19/009,555

(22) Filed: Jan. 3, 2025

Related U.S. Application Data
(60) Provisional application No. 63/617,263, filed on Jan. 3, 2024.

(51) Int. Cl. G01N 33/68; G01N 33/543; G01N 33/569

(52) U.S. Cl. CPC G01N 33/6854; G01N 33/54313; G01N 33/56977; G01N 2333/70539

(57) ABSTRACT

Described herein are materials and methods for determining whether antibody reactivity against an HLA Class 2 antigen is dependent upon the presence of a peptide bound to the peptide binding groove thereof.

### CROSS-REFERENCE TO RELATED APPLICATION

This application claims the priority benefit of U.S. Patent Application No. 63/617,263 filed Jan. 3, 2024, which is incorporated herein by reference in its entirety.

### INCORPORATION BY REFERENCE OF MATERIAL SUBMITTED ELECTRONICALLY

Incorporated by reference in its entirety is a computer-readable sequence listing submitted concurrently herewith and identified as follows: Filename: 59837A_SeqListing.xml; Size: 42,946 bytes; Created: Dec. 24, 2024.

### FIELD OF THE INVENTION

The present invention relates to Human Leukocyte Antigen (HLA) testing such as for determining suitability for transplantation.

### BACKGROUND

Transplant rejection occurs when the immune system of the recipient of a transplant, particularly antibodies produced by the recipient, attacks the transplanted organ or tissue. The recipient's immune system recognizes the transplanted organ as foreign tissue and attempts to destroy it (host vs graft reaction). Rejection also occurs when the transplanted organ consists of donor lymphocytes or progenitor stem cells, which may generate an immune response to the recipient tissues (graft vs. host disease). Chronic rejection is a term used to describe all long-term loss of function in organ transplants associated with chronic alloreactive immune response. Chronic rejection usually leads to graft failure and a need for a new transplanted organ about a decade after the initial transplant. Human leukocyte antigens (HLA) are one type of target molecule within a transplanted organ to which the recipient's immune system responds resulting in transplant rejection. The HLA system is highly polymorphic in nature.

It is a standard practice in the transplant field to evaluate all potential recipients for HLA antibodies by testing against a panel of HLA antigens selected to represent an approximate distribution in the human population. In vitro testing determines the specificity of HLA antibodies present in a patient's blood. The detection of HLA antibodies in a patient's serum that are directed against specific HLA alleles present in a potential donor (donor-specific antibody; DSA) is an indication of risk for graft rejection or failure.

Alloantibodies, particularly when donor specific, are one of the most important factors that cause both early and late graft rejection. Despite improvements in transplantation outcomes, antibody-mediated rejection (AMR) remains a significant impediment to long term graft survival and is associated with increased morbidity, mortality, and costs (Colvin, ASN 18 (4): 1046-1056, 2007).

The presence of HLA antibody is widely believed to be one of the major elements contributing to humoral graft rejections. Transplant recipients with high levels of DSA are at risk for with early graft rejection and failure. Elevated donor specific HLA antibodies in organ recipients either before and/or after allograft transplantation has been associated with acute and chronic AMR and decreased long term graft survival.

HLA molecules exist in two forms; HLA class I molecules consist of a 45-kDa glycoprotein (heavy chain) non covalently associated with a 12-kDa polypeptide, β2-microglobulin (β2m). Association of β2m with newly synthesized class I heavy chains is required in order for the HLA molecule to be expressed and present the peptide (Krangel et al., Cell 18:979, 1979). However, 2m free class I heavy chains were identified on activated T lymphocytes (Schnabl et al., J. Exp. Med. 171:1431, 1990) and other cell surfaces (Bix & Raulet, J. Exp. Med. 176 (3) 829-34, 1992).

HLA class II molecules are heterodimers formed by noncovalent association of two glycosylated polypeptide chains referred to as alpha and beta chains. The a subunit is 33 kDa and the β subunit is 28 kDa, and both chains are transmembrane polypeptides that have the same overall structure. The non-polymorphic α chain is encoded by the DRA gene and can associate with various polymorphic β chains that are encoded by a specific DRB gene. In addition, the DP and DQ HLA gene families each have one gene that encodes an α chain and a β chain. (Reviewed in Choo, Yonsei Med. J. 48:11-23, 2007).

The HLA gene cluster is located within the Major Histocompatibility Complex (MHC) region on the short arm of human chromosome 6. HLA Class I molecules are comprised of a heavy chain and a β2 microglobulin, while HLA Class II molecules are assembled by the α and β chains. However, both types of molecules have similar tertiary structure with the two a helix sidewalls and a β sheet creating a natural grove to bind peptides from both self and non-self-antigens.

The HLA system contains the most polymorphic gene cluster in the entire human genome and is located on the short arm of human chromosome 6. Currently there are 25,019 Class I alleles and 10,201 Class II HLA alleles as reported by the IMGT/HLA Database. The different alleles account for a great deal of diversity of HLA antigens to present as many peptides as possible to TCR's and elicit a specific immune response against most antigens. Due to their polymorphic nature HLA antigens can serve as immunogens and elicit a cellular and humoral alloimmune responses as well. The most common mechanisms of inducing alloimmunity are via pregnancies, transfusions, and transplants. Among transplant recipients, an alloantibody response to a donor's HLA antigens has been shown to be a cause of allograft failure. (Patel and Terasaki, NEJM 280:735, 1969) Pre-transplant, candidates are routinely screened for the presence of Class I and Class II antibodies with solid phase assays utilizing beads coated with single HLA proteins. Normally, DRβ chains pair with a specific, and minimally polymorphic, DRα chain. The same is true for DQ and DP alpha and beta chains, however, in contrast to DRα, both DQA and DPA are highly polymorphic. Importantly, further diversity is generated when DRα pairs with DQβ forming unique, cross-locus heterodimers.

Antibodies to HLA represent a barrier for patients awaiting transplantation¹. Based on reactivity patterns in single antigen bead (SAB) assays, various epitope matching algorithms have been proposed to improve transplant outcomes². However, some antibody reactivities cannot be explained by amino acid motifs, leading to uncertainty about their clinical relevance. Antibodies against DQβ0603:DQα0103, present in some candidates, represent one such example.

The presence of DSA has represented a challenge to solid organ transplantation since it was first demonstrated in 1969 with the cytotoxicity crossmatch¹. Over the years, various cell-based assays, with improved sensitivity, were introduced to improve outcomes^{3,4}. When a multiplex, bead-based assay that used single HLA antigens was introduced in 2003⁵, the HLA antibody profile of patients could be clearly determined even when patients possessed a complex combination of HLA antibodies. The SAB assay has now become an integral part of determining donor-recipient compatibility and has resulted in significant changes to the allocation algorithm for solid organ transplantation⁶. In the United States, UNOS (United Network for Organ Sharing) has established the Calculated Panel Reactive Antibody (cPRA). The cPRA is based on the recipient's HLA antibody profile derived from solid phase assays and the frequency of the selected HLA antigens in a donor population. The cPRA has become an important factor used in renal organ allocation⁶ in the US. In addition to cPRA, refinements to HLA antibody assessments have permitted the concept of the Virtual Crossmatch (VXM). The VXM has become common practice at many centers and has been utilized in lieu of a physical cell-based crossmatch⁷. The viability of both the cPRA and VXM are dependent upon accurate HLA antibody assignments obtained from SAB assays. However, over the years, many publications have established that SAB assays can exhibit discordant results when compared to cell-based crossmatch methods⁸. Studies have demonstrated that denatured HLA antigens on the beads can expose cryptic epitopes that may explain some discordant results^{9,10}. Nevertheless, there are other discordant results, especially for Class II antibody reactivity, that are not completely understood⁸. Determining the cause of these discordant antibody reactivities can facilitate the interpretation of a patient's antibody profile and thereby improve the accuracy of the cPRA and VXM.

Alloantibodies, directed against the HLA DQ molecule (α and β chains) have been frequently observed in transplant recipients¹¹. De novo, donor-specific HLA-DQ antibodies have been shown to be the most prevalent HLA antibody formed, post-transplant, in DSA positive recipients and has been significantly associated with chronic AMR and lower overall graft survival¹². However, the observations that DQ antibodies can be frequently detected in non-sensitized patients and may not produce a positive cell-based XM have generated confusion and concern related to the interpretation of results and how best to utilize the information for patient management.

One explanation for discordance between cell based and bead-based assays is the fact that HLA antigen bearing beads display comparable amounts of DR and DQ molecules while cells express significant lower amounts of DQ antigens on their surface compared to DR13. This difference leads to increased sensitivity of bead-based assays for detecting HLA DQ antibodies. However, this fact cannot explain why non-sensitized patients produced such HLA DQ-reactive antibodies. Thus, investigating the formation and the nature of DQ antibody production could provide useful information for solid organ allocation and AMR treatment.

Of interest to the present invention is the disclosure of Bray et al. US 2020/0072831 which is directed to methods of detecting alloantibodies to HLA Class II antigens and relates to methods and materials for incorporating Class II-associated invariant chain peptide (CLIP) peptides into the peptide binding groove of HLA Class II antigens and using such antigens for the detection of alloantibodies.

Many new HLA alleles have been identified in recent years due to the advances in molecular typing technology, but the serological reactivity of a majority of these alleles has not been determined. Various algorithms have been developed that aim to address this problem, and to improve the accuracy of the VXM by defining antibody reactive epitopes based on HLA protein sequence alignment and 3D structure modeling^{18,19}. Antibody reactivities that could not be assigned to epitopes by such algorithms were frequently questioned for their validity²⁰.

As a consequence of these discordant results there remains a need in the art for improved methods for determining compatibility between donor tissues and recipients.

### SUMMARY

The present invention relates to the showing that peptides derived from amino acid 119-148 of HLA Class I heavy chain are bound to DQβ0603:DQα0103 proteins and contribute to the antibody reactivity through an HLA-DM-dependent process. Moreover, antibody reactivity was impacted by the specific amino acid sequence presented. The present invention relates to the observation that polymorphic peptides including but not limited to HLA Class I peptides, bound to HLA Class II proteins, can directly or indirectly be part of the antibody binding epitope. These findings have potential important implications for the field of transplant immunology and for understanding of adaptive immunity.

The present invention therefore relates to the existence of a type of antibody epitope not previously recognized. This unique epitope is based in part on the peptides bound to the Major Histocompatibility Complex (MHC) binding groove of HLA Class II antigens. The findings are based on the extensive investigation of twenty-one DQβ0603:DQα0103 reactive patient sera. The peptides bound to the DQβ0603:DQα0103 antigen in the study were found to be mainly derived from a polymorphic region of HLA Class I proteins, and these twenty-one sera recognized different portions of the intra-locus or inter-locus polymorphic peptides in association with the DQβ0603:DQα0103 dimer to collectively form the antibody reactive epitope. The participation of the polymorphic residue can either be direct through interaction with the exposed sidechain or indirect through peptide interaction with the base of the binding groove altering the structure of the protein and thus influence antibody binding²¹.

Patient sera that reacted to the DQβ0603:DQα0103 heterodimer without cross-reacting with other HLA DQ antigens was investigated. This specific and unique reactivity was chosen since it cannot be explained based on shared protein sequences among other DQ antigens. Given that no obvious amino acid sequence motif could explain the reactivity, it is reasoned that antibodies specific to the DQβ0603:DQα0103 protein might be recognizing an epitope that involves bound peptides¹⁴.

It was discovered that polymorphic, either inter-locus or intra-locus, peptides derived from the HLA Class I heavy chain were loaded into the DQβ0603:DQα0103 peptide binding groove through an HLA-DM-dependent process to form the epitope recognized by the DQβ0603:DQα0103 specific antibodies. Furthermore, the polymorphism of the peptide sequences bound to the groove of the protein was found to affect the avidity of the antibodies to the DQβ0603:DQα0103 protein.

The loading and binding of peptides to Class II molecules is controlled through an HLA DM dependent process via removal of CLIP (Class II Invariant Chain peptide) from the peptide binding groove of Class II proteins¹⁵. This action facilitates the loading of alternative peptides derived from internalized pathogens or endogenous proteins. It has been reported that the HLA DR3 protein expressed in the B cell line 721.174 (a cell line that has a known deletion of all expressible genes in the class II region) failed to react with a DR3 monoclonal antibody¹⁶. However, transfection of HLA DMA and DMB genes into 721.174 (DR3) cells restored the reactivity of the monoclonal antibody with the DR3 protein¹⁷. It is speculated that DQβ0603:DQα0103 specific sera might be interacting with a DQβ0603:DQα0103/peptide complex through the same mechanism. Similar observations have been reported with certain anti-DQ monoclonal antibodies failed to recognize the DQ proteins expressed on either mouse L cell or human HeLa cell transfectants most likely due to the lack of appropriate peptides bound to the binding groove of the DQ protein16. Patient sera that react to the DQβ0603:DQα0103 heterodimer without cross-reacting with other HLA DQ antigens are frequently observed. This specific and unique reactivity cannot be explained by either unique or shared protein sequences among other DQ antigens. Given that no obvious amino acid sequence motif accounts for the reactivity, it is believed that DQβ0603:DQα0103 specific sera might be interacting with a DQβ0603:DQα0103/peptide complex through a similar peptide-dependent mechanism.

According to one aspect of the invention a method is provided of determining whether antibody reactivity against an HLA Class 2 antigen is dependent upon the presence of a peptide, including the presence of a specific peptide, bound to the peptide binding groove of said HLA Class 2 antigen such as but not limited to an HLA Class 1 peptide bound to the peptide binding groove of said HLA Class 2 antigen said HLA Class 2 antigen comprising the steps of: creating a panel comprising at least two of the same HLA Class 2 antigens with different peptides bound to the peptide binding grooves of said HLA Class 2 antigens, contacting a sample containing antibodies specific for at least one HLA Class 2 antigen and determining whether an antibody which binds to a first HLA Class 2 antigen with a first peptide bound to the peptide binding groove of said HLA Class 2 antigen binds to said first HLA Class 2 antigen with a second peptide bound to the peptide binding groove of said HLA Class 2 antigen. More specifically a method is provided whether an antibody which binds to a first HLA Class 2 antigen with a first HLA Class 1 peptide bound thereto binds to said first HLA Class 2 antigen with a second HLA Class 1 peptide bound thereto.

According to one example, the peptide is derived from amino acids 119-148 of the HLA Class 1 heavy chain but other polymorphic peptides expressed from the genetic background of the cells from which the DQ antigens are produced are contemplated. Also, the HLA Class 2 antigen can be a DQ antigen and more specifically a DQβ0603:DQα0103 antigen.

Also provided are solid substrates having an HLA Class 2 antigen bound to the peptide binding groove of said HLA Class 2 antigen thereto wherein the HLA Class 2 antigen has an HLA Class 1 peptide bound to the peptide binding groove of said HLA Class 2 antigen thereof. Such substrates can comprise a single antigen bead (SAB).

Also provided are assay devices comprising a solid phase array of at least two of the same HLA Class 2 antigens with different peptides such as but not limited to HLA Class 1 peptides bound to the peptide binding grooves of said HLA Class 2 antigens. Particularly preferred are assays comprising multiple assay beads wherein at least two beads present of the same HLA Class 2 antigens with different peptides, including but not limited to HLA Class 1 peptides bound to the peptide binding groove of said HLA Class 2 antigens. According to an alternative embodiment, assay devices are provided comprising a solid phase array of at least two of the same HLA Class 2 antigens wherein a first HLA Class 2 antigen has a peptide bound to the peptide binding groove of said HLA Class 2 antigen, such as but not limited to an HLA Class 1 peptide bound thereto and a second HLA Class 2 antigen has no peptide bound thereto.

Also provided are methods of tissue typing for determining transplant compatibility comprising contacting a serum sample from a potential transplant recipient to a solid substrate having an HLA Class 2 antigen bound thereto wherein the HLA Class 2 antigen has a peptide bound thereto such as an HLA Class 1 peptide bound to the peptide binding groove of said HLA Class 2 antigen thereof.

According to one aspect of this embodiment the HLA Class 1 peptide is derived from amino acids 119-148 of the HLA Class 1 heavy chain. Further preferred are embodiments wherein the HLA Class 2 antigen is a DQ antigen and in particular wherein the HLA Class 2 antigen is an DQβ0603:DQα0103 antigen.

Also provided is a method of detecting DQβ0603:DQα0103 specific antibodies comprising contacting a sample suspected of containing said antibodies with an HLA Class 2 antigen having a peptide such as but not limited to an HLA Class 1 peptide bound to the peptide binding groove of said HLA Class 2 antigen. Particularly preferred is a method wherein the sample is contacted with at least two of the same HLA Class 2 antigens wherein a first HLA Class 2 antigen has first peptide such as but not limited to an HLA Class 1 peptide bound to the peptide binding groove of said HLA Class 2 antigen thereto and a second HLA Class 2 antigen has a different peptide bound to the peptide binding groove of said HLA Class 2 antigen thereof.

### BRIEF DESCRIPTION OF THE DRAWINGS

FIGS. 1A-1B. Reactions of DQβ0603:DQα0103 specific sera to the DQβ0603:DQα0103 protein purified from five different B cell lines.

Antibody reactivity was measured as MFI using the LABScreen SAB assay protocol. The data presented here were the average MFI values from triplicate tests (n=3). a. with six DQβ0603:DQα0103 specific sera and a weak positive control serum, PC #1. b. with four monoclonal antibodies. FM5148 and Tu169 are specific for a monomorphic epitope on DQα, Genox 3.53 is specific for DQα01 and HL-37 is specific for DQβ01,03 and a strong positive control serum, PC #2.

FIGS. 2A-2D. Reactions of DQβ0603:DQα0103 specific sera to the recombinant DQβ0603:DQα0103 protein from different host cell lines.

The antibody reactivity of DQβ0603:DQα0103 protein extracted from transfectants of six different host cell lines were measured as MFI using the LABScreen SAB assay protocol. The data presented here were the average MFI values from triplicate tests (n=3). a. with six DQβ0603:DQα0103 specific sera and a weak positive control serum, PC #1. b. with four monoclonal antibodies. FM5148 and Tu169 are specific for a monomorphic epitope on DQα, Genox 3.53 is specific for DQα01 and HL-37 is specific for DQβ01,03 and a strong positive control serum, PC #2. c. Staining patterns of FR3315 (anti-HLA Class I), FM5148 (anti-HLA DQα) and S1 on T2 or T2DM were shown in re line; the isotype control was shown as filled blue histogram. d. Staining patterns of FR3315 (anti-HLA Class I), FM5148 (anti-HLA DQα) and S1 on LCLKO or LCL3023 were shown in red line and HLA negative serum staining was shown as filled grey histogram.

FIGS. 3A-3D. Reactions of DQβ0603:DQα0103 specific sera to recombinant DQβ0603:DQα0103 protein purified from LCLKO with A*02:01 or C*16:01 gene.

The antibody reactivity of DQβ0603:DQα0103 protein extracted from co-transfectants of DQβ1*06:03, DQA1*01:03, A*02:01 or DQβ1*06:03, DQA1*01:03, C*16:01 of LCLKO host cell line, were measured as MFI using the LABScreen SAB assay protocol. The data presented here were the average MFI values from triplicate tests (n=3). a and c. with six DQβ0603:DQα0103 specific sera and a weak positive control serum, PC #1. b and d. with four monoclonal antibodies, FM5148 and Tu169 specific for a monomorphic epitope on DQα, Genox 3.53 specific for DQα01 and HL-37 specific for DQβ01,03, and a strong positive control serum, PC #2.

FIGS. 4A-4G. Avidity of DQβ0603:DQα0103 sera to DQβ0603:DQα0103 protein loaded with various HLA Class I heavy chain peptides.

DQβ0603:DQα0103 protein from BLS transfectant was loaded with various HLA Class I heavy chain peptides and then used to evaluate the serum reactivity. The antibody reactivity to each peptide loaded antigen was calculated by subtracting the reactivity of the same antigen incubated without the peptide. The values were adjusted to 0 when lower than 0. Successful peptide loading for all peptides was demonstrated by the strong SAPE interaction with biotin at the N-terminal of the peptides (4g).

### DETAILED DESCRIPTION

The present invention relates to the existence of a type of antibody epitope not previously recognized. According to one aspect of the invention it has been found that human HLA Class II antibody reactivity can be influenced by polymorphic peptides, derived from the HLA Class I proteins, bound to HLA Class II peptide binding groove. It is anticipated that other similar types of antibodies might be influenced by peptides derived from other proteins that are also polymorphic in nature. This discovery has multiple implications. For transplantation, it is critical to correctly identify antibody-binding epitopes based on SAB data and HLA typing in order to facilitate an accurate VXM. Since current SAB technologies are dependent on recombinant HLA proteins generated in the context of a limited HLA Class I background, these technologies might underestimate peptide dependence of HLA antibody reactivity in a given patient that has been immunized in the setting of a different HLA Class I background.

Current molecular based analysis of graft biopsies suggests that in up to 50% of patients with AMR and documented evidence of graft damaged as assessed by elevated donor derived cell-free DNA, do not have a detectable DSA based on current SAB assays^{22,23}. It has been suggested that non-HLA antibodies are responsible for the AMR in these cases but it is believed that a portion of these patients may have peptide-dependent HLA that cannot be detected with the current SAB assays. Accordingly, as one aspect of the invention SAB and other assays are provided in which HLA Class 2 antigens are produced having HLA Class 1 peptides bound thereto. In particular, HLA Class 1 peptides derived from amino acids 119-148 of the HLA Class 1 heavy chain are preferred for incorporation with said HLA Class 2 antigens.

Exemplary solid-phase assays such as assays of the invention may use solid substrates such as microparticles, magnetic particles such as ferromagnetic beads and paramagnetic beads, microtiter plates, membranes, filters, glass, metal, metal-alloy, anopol, polymers, nylon, plastic, or microarrays such as protein chips. Microarrays may be of any material such as glass or silica. Binding on a microtiter plate may be detected using ELISA assays, RIA assays or other immunosorbent sandwich assays. Binding on a filter may be detected using immunoblotting techniques.

The solid-phase assays of the invention may be carried out with microparticles, microbeads, magnetic beads, beads, or microspheres of any material, e.g., silica, gold, latex, polymers such as polystyrene, polysulfone and polyethyl, or hydrogel. Additional exemplary microparticles are encoded with the dyes and the antigens are immobilized to the encoded microparticles. The microparticles used in the methods of the invention are commercially available from sources such from Luminex Inc., Invitrogen (Carlsbad, CA), Polysciences Inc. (Warrington, PA) and Bangs Laboratories (Fishers, IN) to name a few.

The microparticles of the invention may comprise a detectable label or another identifying characteristic. The microparticles may comprise a single fluorescent dye or multiple fluorescent dyes. In one embodiment, the microparticles are internally labeled with fluorescent dyes and contain surface carboxyl groups for covalent attachment of biomolecules. In another embodiment, the microparticles are internally labeled with fluorescent dyes and contain a surface layer of Avidin for near covalent binding of biotin and biotinylated ligands. In another embodiment, the microparticles may comprise a combination of different dyes, such as a fluorescent and a non-fluorescent dye. For example, the microparticles may be labeled with E)-5-[-[2-methoxycarbonyl) ethenyl]cytidine, which is a nonfluorescent molecule, that when subjected to ultraviolet (UV) irradiation yields a single product, 3-β-D-ribofuranosyl-2,7-dioxopyrido[2,3-d]pyrimidine, which displays a strong fluorescence signal. In another embodiment, the microparticles may comprise bar codes as an identifiable characteristic as described in U.S. Patent Publication No. 2007/0037195.

In another embodiment, the microparticles may be nanocrystals or quantum dots. These nanocrystals are substances that absorb photons of light, then re-emit photons at a different wavelength (fluorophores). In addition, additional florescent labels, or secondary antibodies may be conjugated to the nanocrystals. These nanocrystals are commercially available from sources such as Invitrogen and Evident Technologies (Troy, NY).

The invention can be carried out with any system that detects the identifiable characteristic or label, such as FLOW cytometry. Detection of fluorescent labels may also be carried out using a microscope or camera that will read the image on the microparticles, such as the Bioarray BeadChip (Bioarray Solutions, Ltd., Warren, NJ). The BeadChip format combines microparticle (“bead”) chemistry with semiconductor wafer processing in which binding to the microparticle is recorded using an optical microscope and camera.

The invention also may be carried out using column chromatography, affinity chromatography, thin layer chromatography, liquid-phase immunodiagnostic (LIPA) assays, liquid-phase chemiluminescent ELISA and liquid-phase immunoradiometric (IRMA) to name a few.

Biological samples include whole blood, blood derivatives, red blood cell concentrates, plasma, serum, fresh frozen plasma, whole blood derived platelet concentrates, apheresis platelets, pooled platelets, intravenous gamma-globulin, cryoprecipitate, cerebrospinal fluid, tissues, and cells such as epithelial cells, such as those collected from the bucal cavity, stem cells, leukocytes, neutrophils, and granulocytes. The biological samples may be obtained from a human donor of tissue or cells intended for transplantation or a human donor of blood or blood derivatives intended for transfusion. The biological sample may be obtained from a healthy bone marrow donor or a subject of a paternity test. The biological sample may also be obtained from a human subject that is an intended recipient of a transplant or transfusion, or the human subject that is donating the tissue or organ intended for transplantation or transfusion. Alternatively, the biological sample may be obtained directly from tissues or cells that are intended for transplantation in a human recipient. In addition, the biological sample may be obtained from blood or blood derivatives that are intended for transfusion in a human recipient.

The antibodies of the invention may be polyclonal antibodies, monoclonal antibodies, antibody fragments which retain their ability to bind their unique epitope (e.g., Fv, Fab and F (ab) 2 fragments), single chain antibodies and human or humanized antibodies. Antibodies may be generated by techniques standard in the art using an antigenic HLA epitope. See, e.g., Kohler et al., Nature, 256:495-497 (1975), Brodeur et al., Monoclonal Antibody Production Techniques and Applications, pp. 51-63 (Marcel Dekker, Inc., New York, 1987). Antibody molecules of the present invention include the classes of IgG (as well as subtypes lgG 1, lgG 2a, and IgG2b), IgM, IgA, IgD, and IgE.

The antibodies of the invention may be labeled for detection of binding within the biological sample. The antibodies may comprise a radioactive label such as ³H, ¹⁴C, ³²P, ³⁵S, or ¹²⁵I. In addition, the labels may be a fluorescent or chemiluminescent compound, such as fluorescein isothiocyanate, phycoerythrin, rhodamine, or luciferin. The labels may be enzymes such as alkaline phosphatase, β-galactosidase, biotin and avidin or horseradish peroxidase (Bayer et al., Meth. Enz., 184:138-163 (1990)).

The antibodies specific for HLA antigens may be attached to solid substrates such as membranes, beads, filters, glass, silicon, metal, metal-alloy, anopore, polymeric, nylon or plastic for detection of antigens in a biological sample.

Specific binding of an antibody to an HLA antigen within a biological sample may be carried out using Western blot analysis with immunoblotting, immunocytochemistry, immunohistochemistry, dot blot analysis, flow cytometry, ELISA assays or RIA assays. These techniques and other approaches are conventional in the art (See Sambrook et al., Molecular Cloning: A Laboratory Manual, cold Springs Harbor Laboratories (New York, 1989).

Of interest to the present invention are assay methods making use of flow cytometry. Wilson et al., J. Immunol. Methods 107:231-237 (1988) disclose the use of polyacrylamide microspheres coupled with cell membrane proteins in immunofluorescence assays for antibodies to membrane-associated antigens. The method is said to make possible the rapid flow cytometric analysis of plasma membrane antigens from cell populations that would otherwise be unsuitable for use in flow cytometry. Scillian et al., Blood 73:2041-2048 (1989) disclose the use of immunoreactive beads in flow cytometric assays for detection of antibodies to HIV. Frengen et al., Clin. Chem. 40/3:420-425 (1994) disclose the use of flow cytometry for particle-based immunoassays of ce-fetoprotein (AFP). This reference further reports the ability of serum factors to cross-link labeled mouse monoclonal antibodies of irrelevant specificity to different particle types coated with various immunoglobulins.

Flow cytometry methods using lymphocytes are also known but suffer with difficulties because of the activity of auto-antibodies. See Shroyer et al., Transplantation 59:626-630. Moreover, when using flow cytometry with lymphocytes, use of ten or more different lymphocytes tends to result in confusing signals. As a consequence, studies using lymphocytes have been limited by presenting a small panel of HLA antigens that do not effectively simulate the distribution of HLA antigens in a normal human population.

Sumitran-Karuppan et al., Transplantation 61:1539-1545 (1996) discloses the use of magnetic beads which use an anti-HLA capture antibody to immobilize a variety of soluble HLA antigens pooled from 80 to 100 individuals on each bead. The beads can then be directly added to patient serum for efficient absorption of HLA antibodies. The reference discloses visualization of antibody binding to the antigen-coated beads using flow cytometry. The reference suggests that this development will allow testing for antibody specificity for crossmatching purposes and for the screening of panel-reactive antibodies. The methods of Sumitran-Karuppan are limited, however, because the pooling of antigens causes sensitivity to certain rare HLA antigens. Moreover, the method is not capable of detecting the percentage of PRA.

The invention also provides for kits to carry out the methods of the invention. In particular, the invention provides for kit for determining the presence of antibodies in serum of a subject against HLA Class II antigens comprising a first collection of solid-phase substrates wherein each solid-phase substrate is coated with different purified HLA antigens to represent the HLA antigen population of a single cell line such that said collection simulates the distribution of HLA antigens in a normal human. The antigens provided in the kit may be conjugated to solid substrates in the kit. Alternatively, the kit comprises solid substrates and antigens and the skilled artisan can conjugate the antigens to the solid substrates allowing for optimization of the antigens used in the assay. The kits may also comprise the reagents necessary to detect and measure antibodies, such as HLA antibodies for use as a positive control.

The HLA antigens comprise Class II HLA antigens (e.g., wherein the HLA antigens are selected such that the HLA antigens presented on the solid phase substrate comprise Class II HLA antigens so as to simulate the distribution of Class II HLA antigens in a normal human population). In some embodiments, the HLA antigens also comprise HLA Class 1 antigens.

The kits useful herein may further comprise any components necessary to carry out the detection assays that are conventional in the art. For example, the kits may comprise buffers, loading dyes, gels such as polyacrylamide gels and molecular weight markers for preparing SDS-PAGE gels to carry out Western blots. The kits may also comprise filters, membranes blocking buffers, control buffers, isotype control antibodies, wash buffers or buffers and reagents for detection to carry out immunoblotting or dot blotting analysis such as labeled secondary antibodies. The kit may also comprise fixing reagents, blocking buffers, control buffers, wash buffers, staining dyes and detection reagents including anti-idiospecific antibodies. Furthermore, the kits may comprise the necessary reagents and tools to carryout flow cytometry, ELISA assays, RIA assays or microtoxicity assays.

In some embodiments, the detecting step comprises detecting labeled ligand bound to the complex to determine the presence or absence of antibodies reactive against selected HLA Class II antigens. In some embodiments, detecting of the labeled ligand is carried out by flow cytometry. In some embodiments, the detecting step comprises detecting the presence of the complex using a solid phase immunoassay or a multiplexed bead immunoassay.

The solid-phase substrate can be any solid substrate known in the art. In some embodiments, the solid-phase substrate is selected from the group consisting of microparticle, microbead, magnetic bead, ion torrent bead, flow cytometer bead and an affinity purification column. In some embodiments, the solid-phase substrate is a microbead. In some embodiments, the microbead is a latex microbead. The microbead, in some embodiments, has a diameter ranging from about 2 μm to about 15 μm, inclusive. Microbeads having a diameter of about 2 μm, 3 μm, 4 μm, 5 μm, 6 μm, 7 μm, 8 μm, 9 μm, 10 μm, 11 μm, 12 μm, 13 μm, 14 μm or 15 μm are also contemplated. In some embodiments, at least one microbead presenting Class I HLA antigens is 3 μm is diameter. In some embodiments, at least one microbead presented Class II HLA antigens is 5 μm in diameter. In some embodiments, the microbeads comprise a mixture of 3 μm microbeads presenting Class I HLA antigens and 5 μm microbeads presented Class II HLA antigens.

The present invention relates to the existence of a type of antibody epitope not previously recognized. This unique epitope is based in part on the peptides bound to the Major Histocompatibility Complex (MHC) binding groove of HLA Class II antigens. The findings are based on the extensive investigation of twenty-one DQβ0603:DQα0103 reactive patient sera. The peptides bound to the DQβ0603:DQα0103 antigen in the study were found to be mainly derived from a polymorphic region of HLA Class I proteins, and these twenty-one sera recognized different portions of the intra-locus or inter-locus polymorphic peptides in association with the DQβ0603:DQα0103 dimer to collectively form the antibody reactive epitope. The participation of the polymorphic residue can either be direct through interaction with the exposed sidechain or indirect through peptide interaction with the base of the binding groove altering the structure of the protein and thus influence antibody binding²¹.

Among the twenty-one patients who possessed the DQβ0603:DQα0103 reactivity, twelve had been transplanted with preformed DQβ0603:DQα0103 non-DSA antibody and nine were still awaiting transplant. Most of the twenty-one patients had no documented sensitization event when the antibody was first detected. Hence, these antibodies might also be auto-reactive or related to their disease state. Interestingly, all twenty-one sera identified reacted to epitopes involving HLA Class I peptides, even though the DQβ0603:DQα0103 protein is capable of binding many other peptides (ProteomeXchange Consortium identifier PXD043999).

Without being bound by any specific theory these findings could be interpreted to suggest that B cells, through their B cell receptor (BCR), are able to recognize peptides bound to MHC Class II molecules and through this recognition generate antibodies directed at a specific MHC peptide combination.

The cell-based flow cytometric crossmatch is often used to confirm the reactivity of antibodies detected with the SAB assay. However, since the expression of DQ molecules on the cell surface is significantly lower than that of DR molecules¹³, the detection of such antibodies may be challenging. Moreover, if only a fraction of those DQ molecules have the correct peptide that is necessary for reactivity, this may further add to the frequency of discordant results between cell based and bead-based assays.

The peptides that can bind to DQβ0603:DQα0103 protein are restricted by their anchor residues that fit into the MHC binding groove of the Class II antigens²⁴ and those restricted peptides in turn contribute to the antibody specificity. Changes in the amino acid sequences of the α or β chain that can affect the peptide-binding groove could result in a different peptide repertoire thereby altering the binding specificity of an antibody. This could occur even if two alleles have the same amino acid sequence in the surrounding alpha helix protein structure that is used by epitope prediction algorithms.

According to one aspect of the invention evidence is presented that HLA antibody reactivity with the DQβ0603:DQα0103 protein detected in patient sera both pre- and post-transplant is in part dependent on the presented peptide.

Identification of DQβ0603:DQα0103 specific sera from kidney patients on the wait list of each transplant center were routinely screened on LABScreen Class II single antigen panel (One Lambda Inc., West Hill, CA) and those that reacted only to the DQβ0603:DQα0103 antigen beads among all the DQ antigen beads with the signal greater than 3000 Mean Fluorescence intensity (MFI) were identified for further characterization.

### Cell Lines and Antibodies

EBV transformed B cell lines, 9058, 9060, 9062, 9065, and 9105 from the 10th International Histocompatibility Workshop and Conference (IHWC) Epstein-Barr virus (EBV) transformed B cell lines, LCL3023 and bare lymphocyte syndrome (BLS) B cell line were gifts. K562 and T2 cell lines were purchased from ATCC (Manassas, VA). T2DM, a T2 cell line transfected with HLA-DM (DMA*01:01 and DMB*01:01) cDNA and LCLKO were gifts. T2 was derived from LCL. 17425 which has deletions from DRA to DPB1 in both copies of the chromosomes. LCL302317 is from the LCL. 174 transfected with both HLA-DM and HLA-DRA genomic DNA. LCLKO was derived from LCL721.22126. All BLS, K562, LCL3023, LCLKO, T2 and T2DM cell lines have no detectable expression of HLA Class II on the cell surface. HLA monoclonal antibody FM5148 (DQα) and FR3315 (Class I) were gifts. Genox 3.53 (DQ1), HL-37 (DQ1,3) and MOPC-21 (mouse lgG isotype control) were purchased from Thermo Fisher Scientific (Waltham, MA). Tu169 was purchased from BioLegend (San Diego, CA).

### Transfection and Cell Culture

The DQA1*01:03 and DQβ1*06:03 cDNA sequences as well as HLA-Class I sequences were synthesized according to the published sequences in the IPD-IMGT/HLA Database (https://www.ebi.ac.uk/ipd/imgt/hla/). DQA1*01:03 and DQβ1*06:03 alleles were cloned into the pEF6 Mammalian Expression Vector (Thermo Fisher Scientific, Waltham, MA). HLA Class I gene expression vectors were gifts from One Lambda. Plasmids were co-transfected into human cell lines as mentioned in the text, using a Neon Transfection System (Thermo Fisher Scientific, Waltham, MA) according to the manufacturer's protocol. Cells were pulsed twice with a voltage of 1,100 and a width of 30 ms. Stable cells were established by RPMI-1640 media (Thermo Fisher Scientific, Waltham, MA) with 10% Fetal bovine serum (FBS) (Thermo Fisher Scientific, Waltham, MA) and with corresponding selection antibiotics according to the plasmids used (Thermo Fisher Scientific, Waltham, MA).

### HLA Antibody Assay

HLA Class II antigens were purified by cell lysis and affinity purification from cell pellets using HLA-DQ specific antibody FM5148 and attached to Luminex beads2. Solid-phase bead assay was performed as per the LABScreen antibody detection protocol and analyzed by a LABScan3D flow analyzer (One Lambda, West Hills, CA).

### Flow Cytometry Analysis and Cell Sorting for Transfectant

Monoclonal antibodies were first labeled with Fluorochrome using Alexa Fluor™ Antibody Labeling Kits (ThermoFisher Scientific, Waltham, MA) and then used to label cells for 30 min at 4° C. Following labeling, cells were washed twice with 1×PBS, 0.1% glucose and 2% FBS (cell wash buffer). Flow cytometric analyses were performed in a Quanteon Flow Cytometer (Agilent, Santa Clara, CA). Data was analyzed with FlowJo Software. Stable transfected cells were sorted on a MA900 cell sorter (Sony Biotechnology, San Jose, CA). High immunofluorescent cells were sorted and plated in RPMI-1640 plus 10% FBS with the corresponding selection antibiotics.

Flow Cytometry Analysis with Human Sera

Approximately 10⁶ cells from the stable transfectants were first treat with LIVE/DEAD™ Fixable Near-IR Dead Cell Stain Kit (Thermo Fisher Scientific) according to manufactural procedure. The treated cells were resuspended in 100 μl of cell wash buffer before mixing with 50 μl of test serum, and incubated for 20 minutes at 4° C. After incubation, cells were washed with cell wash buffer and then incubated with biotinylated F (ab′) 2-Goat anti-Human IgG Fc specific antibody (Invitrogen) in 100 μl cell wash buffer for 20 minutes. Samples were washed and incubated with SAPE (Jackson ImmunoResearch Laboratories, West Grove, PA) in 100 μl cell wash buffer for 20 minutes at 4° C. After washing, samples were acquired on a Quanteon Flow Cytometer (Agilent Technologies, Santa Clara, CA). Data was analyzed with FlowJo Software (https://www.flowjo.com/). To check the HLA expression, cells were labeled with fluorochrome-conjugated antibodies, Alexa Fluor® 488 conjugated FR3315 (HLA-Class I) or Alexa Fluor® 647 conjugated FM5148 (HLA-DQα). Alexa Fluor® 488 or 647 conjugated mouse IgG1 isotype controls, clone MOPC-21, were used to establish the baseline for FM5148 or FR3315, respectively. Forty thousand events (ungated) were acquired.

### HLA Peptidome Analysis

Bound HLA antigens from the affinity matrix with monoclonal antibody FM5148 were eluted and processed without the presence of detergents. Peptide samples were analyzed on a NanoLC446 ESI-MS/MS system, a service provided by ProtTech Inc. (Phoenixville, PA).

### Peptide Loading Protocol

HLA Peptides were synthesized by ThermoFisher Scientific (Waltham, MA). Biotinylated HLA peptides (0.5 mM) were incubated with approximately 7,500 beads bearing DQβ0601:DQα0103 protein purified from the BLS transfectant in 50 μl of 150 mM citrate phosphate buffer pH7.2 for 72 hours at 37° C. The reaction mixture was then centrifuged at 14,000×g to pellet the beads, after removing the supernatant, beads were washed twice with 300 μl LABScreen wash buffer and resuspended in 25 μl Phosphate Buffered Saline Solution (Irvine Scientific, Santa Ana, CA) with 2% BSA (Sigma Aldrich, Burlington, MA). Serum reactivity was then measured according to LABScreen SAB assay protocol.

According to the experiments described below polymorphic, either inter-locus or intra-locus, peptides derived from the HLA Class I heavy chain were loaded into the DQβ0603:DQα0103 peptide binding groove through an HLA-DM-dependent process to form the epitope recognized by the DQβ0603:DQα0103 specific antibodies. Furthermore, the polymorphism of the peptide sequences bound to the groove of the protein was found to affect the avidity of the antibodies to the DQβ0603:DQα0103 protein.

Aspects of the invention are further disclosed by the examples provided below.

### Example 1

DQβ0603:DQα0103 Positive Sera have Different Levels of Serological Reactivity to DQβ0603:DQα0103 Proteins Extracted from Different EBV Transformed B Cell Lines

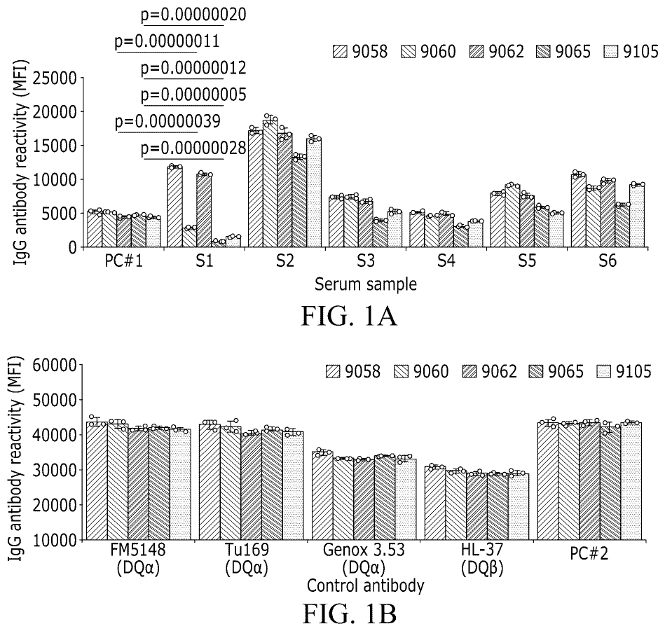

According to this example, six DQβ0603:DQα0103 specific sera (S1-S6) were identified from patients via routine HLA antibody monitoring. These six sera reacted only to the DQβ0603:DQα0103 antigen bead. All other DQ antigen beads on the Class II SAB panel were negative. These six sera were then tested against a bead panel of DQβ0603:DQα0103 proteins purified from five homozygous EBV transformed B cell lines to confirm the presence of DQβ0603:DQα0103 antibody reactivity. All five B cell lines possessed different Class I genotypes (Table 1). Their serological reactivity is shown in FIG. 1A. Surprisingly, the reactivity of the six DQβ0603:DQα0103 specific sera showed at least two distinct profiles to those five DQβ0603:DQα0103 proteins. In contrast, four monoclonal antibodies, among which three recognizing DQα and one recognizing DQβ, and two DQ6 positive control allo-sera (PC #1 and PC #2) showed similar reactivity to the DQβ0603:DQα0103 antigen bearing beads demonstrated that these five DQβ0603:DQα0103 beads have a similar amount of protein on each bead (FIG. 1B).

TABLE 1
The HLA class I genotypes of the EBV-transformed B cells and transfectant hosts

| | | | | Expression | | | |
|---|---|---|---|---|---|---|---|
| Cell | HLA class I type | | | class I | class II | CD74 | DM |
| 9058 | A*02:01 | B*45:01 | C*16:01 | + | + | + | + |
| 9060 | A*01:01 | B*15:01 | C*03:03 | + | + | + | + |
| 9062 | A*02:01 | B*38:01 | C*12:03 | + | + | + | + |
| 9065 | A*03:01 | B*07:02 | C*07:02 | + | + | + | + |
| 9105 | A*01:01 | B*35:02 | C*04:01 | + | + | + | + |
| LCLKO | — | — | C*01:02 | ±* | − | + | + |
| LCL3023 | A*02:01 | B*51:01 | C*01:02 | + | − | + | + |
| T2 | A*02:01 | B*51:01 | C*01:02 | + | − | + | − |
| T2DM | A*02:01 | B*51:01 | C*01:02 | + | − | + | + |
| K562 | A*11:01 | B*18:01 | C*03:04 | − | − | − | − |
| | A*31:01 | B*40:01 | C*05:01 | | | | |
| BLS | A*11:01 | B*41:02 | C*02:02 | + | − | + | − |
| | A*66:01 | B*51:01 | C*17:03 | | | | |

*Very low level of HLA class I protein expression on cell surface

One of the six sera, S1, showed stronger reactivity to the DQβ0603:DQα0103 proteins derived from the 9058 and 9062 cell lines which are both HLA A*02:01. The other three cell lines, 9060, 120 9065 and 9105, are non-A*02:01. The other five sera showed reaction patterns to the five DQβ0603:DQα0103 antigens with slight but distinguishable variations in the relative strength. The significantly different reactivity of S1 to the same protein derived from five different B cell lines indicated that post-translational influence from the host cell lines was likely responsible for the variation.

### Example 2

DQβ0603:DQα0103 Positive Sera Reacted to Recombinant DQβ0603:DQα0103 Proteins Derived from Transfectants of CD74 and DM Positive Host Cell Lines

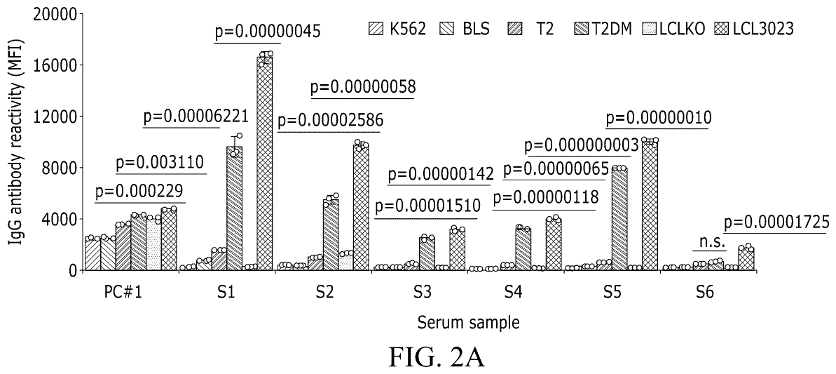

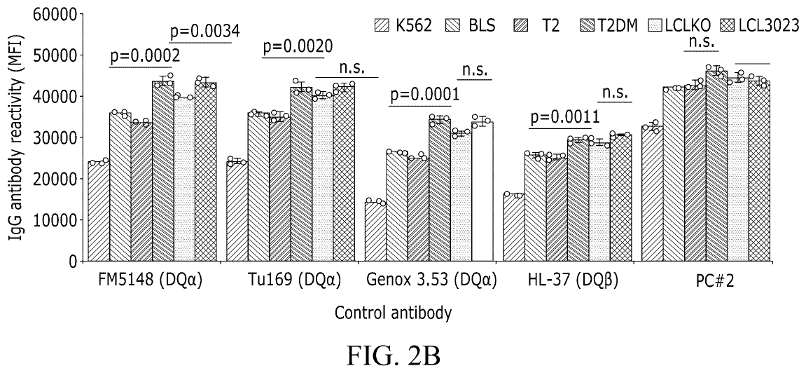

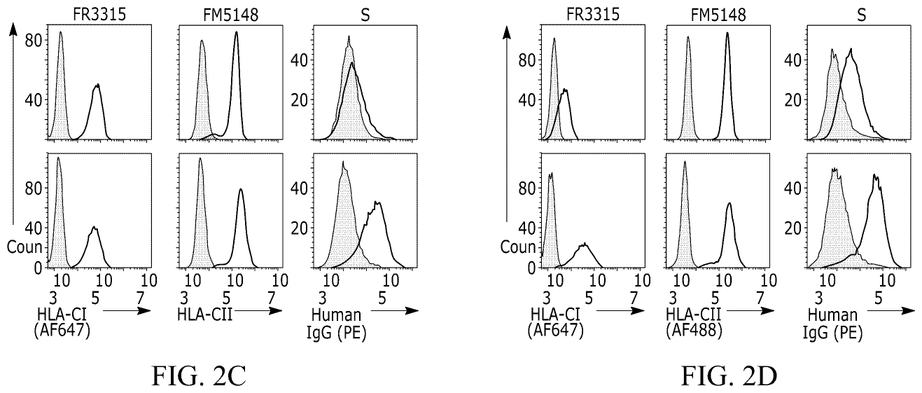

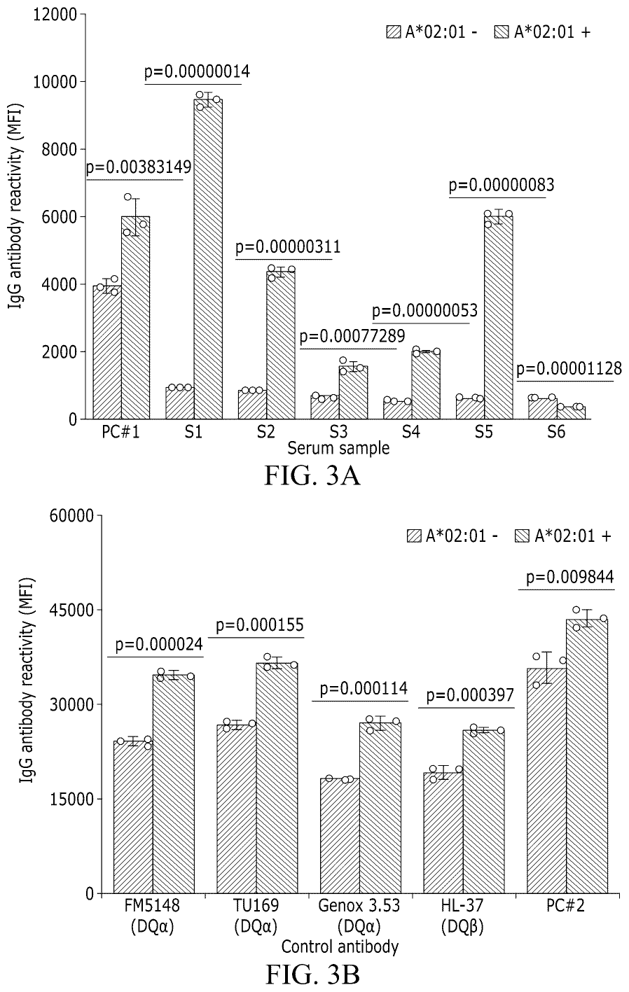

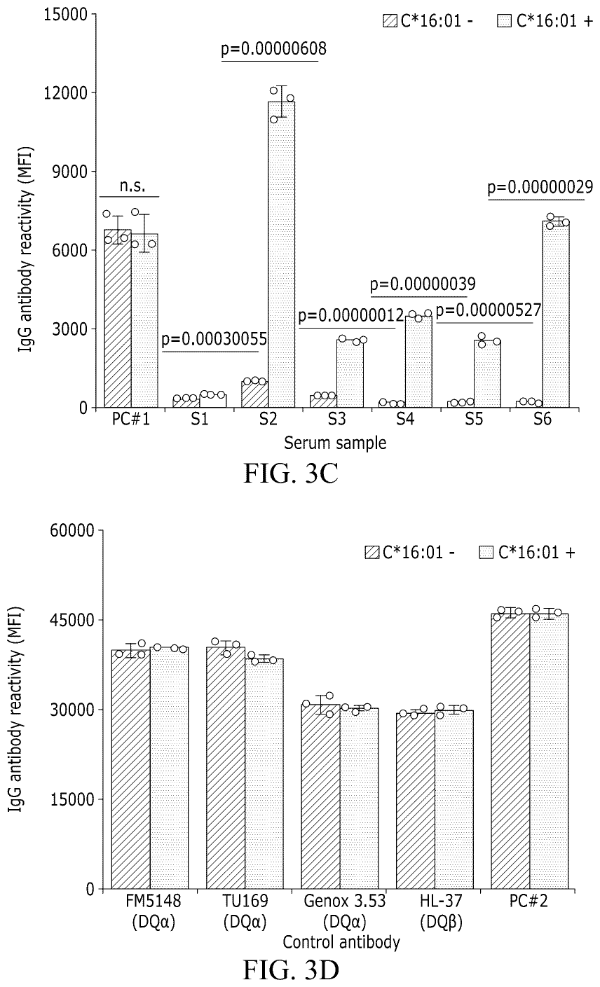

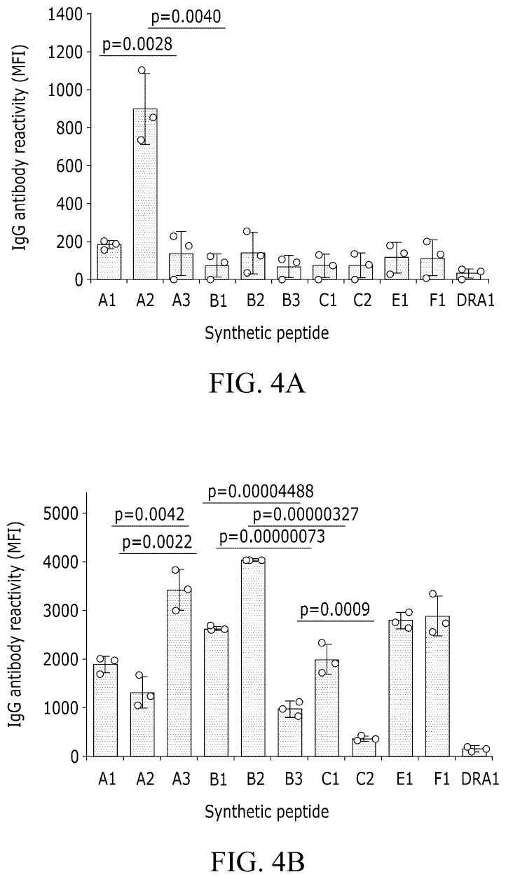

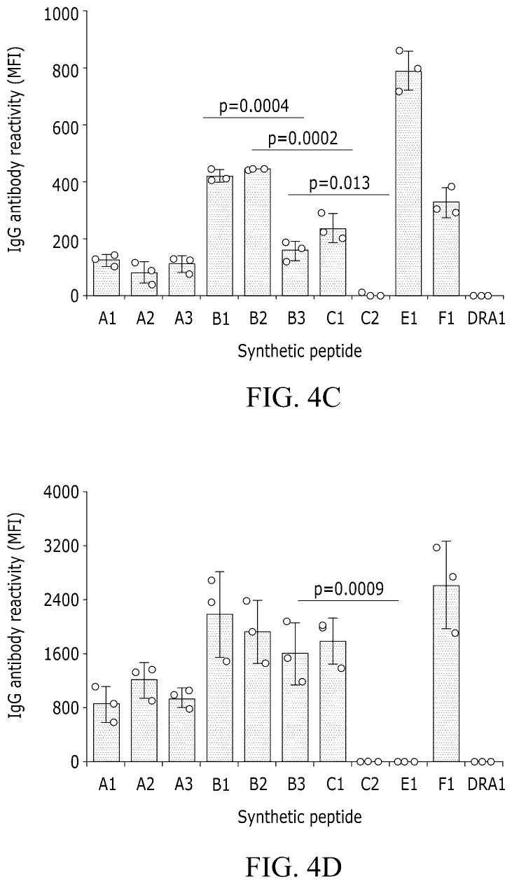

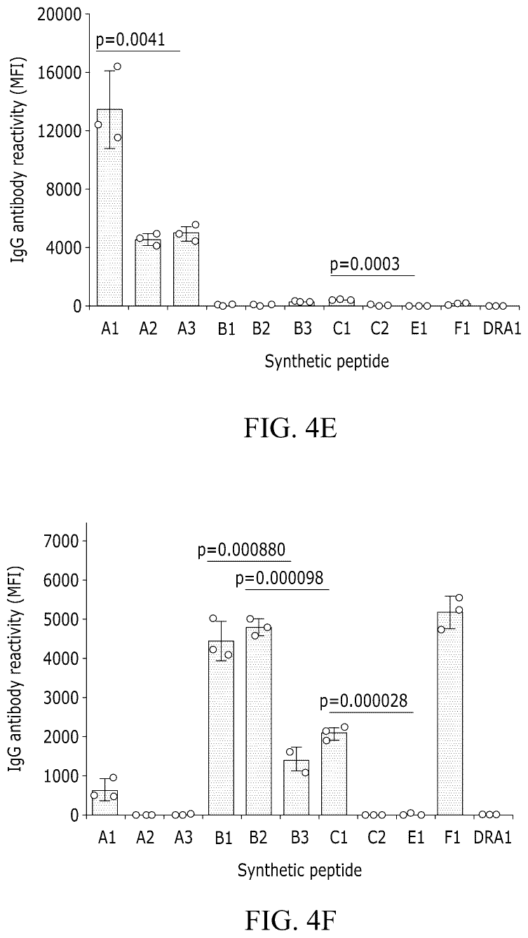

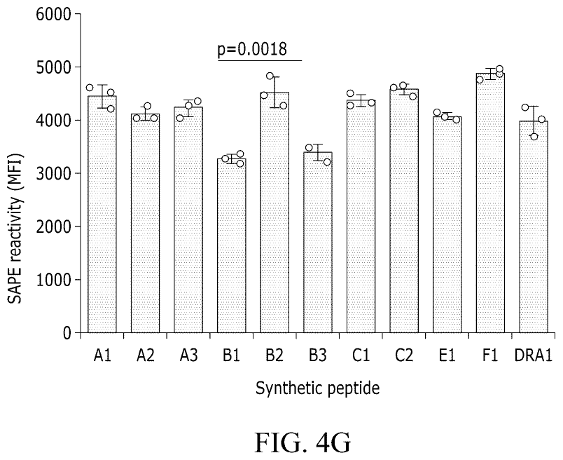

According to this example, the involvement of HLA Class I expression was investigated since S1 preferentially reacted to the DQβ0603:DQα0103 protein isolated from the two HLA129 A*02:01 positive B cell lines. The antibody reactivity of the six DQβ0603:DQα0103 sera was measured against recombinant DQβ0603:DQα0103 proteins extracted from six stable transfectants via the LABScreen SAB assay protocol (FIG. 2A). Importantly, each of the six host cell lines possessed a different genetic background that affects the expression of invariant chain (CD74), HLA-DM or HLA Class I, specifically A*02:01 (Table 1). The same four monoclonal antibodies and two positive control sera were used to assess the amount and the integrity of the recombinant DQβ0603:DQα0103 proteins on the beads. The protein isolated from the CD74-, DM-cell line, K562, has lower reactivity to all four monoclonal antibodies and PC #2 (FIG. 2B) when compared to proteins isolated from DM-BLS or T2 suggesting that CD74 was required to 139 stabilize the DQ protein.

TABLE 2
HLA class I peptides identified from the DQβ0603:DQα0103 protein
peptidome analysis.

| 9060 | Position | | | 9062 | Position | | |
|---|---|---|---|---|---|---|---|
| Allele | start | end | Amino Acid Sequence | Allele | Start | end | Amino Acid Sequence |
| A*01:01 | 119 | 136 | DGKDYIALNEDLRSWTAA | A*02:01 | 129 | 144 | DLRSWTAADMAAQTTK |
| | 129 | 145 | DLRSWTAADMAAQITKR | | 129 | 145 | DLRSWTAADMAAQTTKH |
| | 128 | 144 | EDLRSWTAADMAAQITK | | 128 | 144 | EDLRSWTAADMAAQTTK |
| | 128 | 145 | EDLRSWTAADMAAQITKR | | 128 | 145 | EDLRSWTAADMAAQTTKH |
| | | | | | 128 | 146 | EDLRSWTAADMAAQTTKHK |
| B*15:01 | 128 | 144 | EDLSSWTAADTAAQITQ | B*38:01 | 128 | 148 | EDLSSWTAADTAAQITQRKWE |
| | | | | | 126 | 145 | LNEDLSSWTAADTAAQITQR |
| C*03:03 | 119 | 141 | DGKDYIALNEDLRSWTAADTAAQ | C*:12:03 | 128 | 141 | EDLRSWTAADTAAQ |
| | 128 | 141 | EDLRSWTAADTAAQ | | 128 | 144 | EDLRSWTAADTAAQITQ |
| | 128 | 143 | EDLRSWTAADTAAQIT | | 128 | 145 | EDLRSWTAADTAAQITQR |
| | 128 | 144 | EDLRSWTAADTAAQITQ | | 128 | 148 | EDLRSWTAADTAAQITQRKWE |
| | 128 | 145 | EDLRSWTAADTAAQITQR | | 126 | 141 | LNEDLRSWTAADTAAQ |
| | 128 | 148 | EDLRSWTAADTAAQITQRKWE | | 126 | 144 | LNEDLRSWTAADTAAQITQ |
| | 126 | 141 | LNEDLRSWTAADTAAQ | | | | |
| | 127 | 141 | NEDLRSWTAADTAAQ | | | | |
| E | 128 | 141 | EDLRSWTAVDTAAQ | E | 128 | 141 | EDLRSWTAVDTAAQ |
| | 128 | 144 | EDLRSWTAVDTAAQISE | | 129 | 144 | DLRSWTAVDTAAQISE |
| | 126 | 141 | LNEDLRSWTAVDTAAQ | | | | |
| | 129 | 141 | DLRSWTAVDTAAQ | | | | |
| | 130 | 141 | LRSWTAVDTAAQ | | | | |
| F | 128 | 141 | EDLRSWTAADTVAQ | F | 128 | 141 | EDLRSWTAVDTAAQ |
| | | | | | 129 | 144 | DLRSWTAVDTAAQISE |
| | | | | DRA | 126 | 143 | KPVTTGVSETVFLPREDH |

Five of the six sera, (S1-S5) showed positive reactivity against beads bearing recombinant DQβ0603:DQα0103 protein from T2DM host, but negative on the recombinant DQβ0603:DQα0103 protein from T2 host (FIG. 2A). Since T2 and T2DM differ only on the expression of DM in T2DM, this result would indicate that DM expression is essential for the assembly of the epitope recognized by these DQβ0603:DQα0103 sera. Negative reactions on the recombinant DQβ0603:DQα0103 proteins derived from BLS (CD74+, DM−) or K562 (CD74−, DM−) hosts and positive reaction on the recombinant DQβ0603:DQα0103 protein derived from LCL3023 (CD74+,DM+) host further corroborated the conclusion that CD74 and DM are both required for the expression of the DQβ0603:DQα0103 reactive epitope. The strong reactivity to the recombinant DQβ0603:DQα0103 proteins from the HLA-Class I expressing T2DM and LCL3023 hosts and negative reactivity to the HLA-Class I non-expressing LCLKO host supported the observation shown in FIG. 1A that HLA Class I peptides were participating in forming the epitope recognized by S1-S5.

The transfectants were further tested by flow cytometry for the surface expression and assembly of the DQβ0603:DQα0103 protein with the S1 serum to confirm the reactivity observed on the SAB assay. The expression of the HLA Class I proteins was at a similar level between the T2 and T2DM transfectants, however, the T2DM transfectant has a slightly higher expression of HLA DQ than the T2 transfectant. The positive staining of S1 with the T2DM transfectant, but negative staining with T2 transfectant supports the SAB results suggesting that HLA-DM was essential for the assembly of the epitope recognized by S1 (FIG. 2C). The LCL3023 transfectant had a higher level of expression for HLA Class I and a similar level of HLA DQ expression compared to LCLKO transfectant. However, S1 had stronger staining on LCL3023 transfectant than on the LCLKO transfectant, even though they have a similar level of HLA DQ staining, confirmed the participation of HLA Class I derived peptides in the S1 reactivity (FIG. 2D).

The negative (or very weak) reactivity of S6 on recombinant DQβ0603:DQα0103 protein derived from HLA Class I expressing T2DM and LCL3023, suggests that the peptides derived from the HLA Class I alleles expressed in T2DM or LCL3023 could not provide the epitope recognized by S6.

### Example 3

### Expression of HLA Class I Gene in DQβ1*06:03-DQA1*01:03 Transfectants Rescued the 174 DQβ0603:DQα0103 Antibody Reactive Epitope

According to this example, the HLA A*02:01 or C*16:01 alleles from 9058 cell line (Table 1) were introduced into the LCLKO transfectant expressing DQβ0603:DQα0103 antigen to determine whether peptides derived from C*16:01 or A*02:01 could restore the S6 or S1 epitope reactivity, respectively because the DQβ0603:DQα0103 protein isolated from 9058 B cell line was reactive with S6. The recombinant DQβ0603:DQα0103 proteins were extracted from the co-transfectants and their antibody reactivity compared to the proteins purified from the original LCLKO transfectants. The expression of A*02:01 rescued the serum reactivity of S1 (FIG. 3A), but expression of C*16:01 did not (FIG. 3C). Conversely, incorporation of C*16:01 rescued the serum reactivity of S6 (FIG. 3C), but the incorporation of A*02:01 did not (FIG. 3A), supporting the observation that S6 negative reactivity in FIG. 2 was due to the missing specific HLA Class I peptide in T2DM and LCL3023 hosts and was not caused by other possible mechanisms. Expression of the additional Class I gene in the transfectants didn't affect the reactivity of proteins to the four monoclonal antibodies and PC #2 that recognize the DQα and DQβ chains (FIGS. 3B and 3D). These data strongly implicate the involvement of a specific HLA Class I polymorphic sequence in the DQβ0603:DQα0103 antibody reactivity.

### Example 4

Polymorphic HLA Class I Peptides were Identified to Associate with the DQβ0603:DQα0103 Proteins

According to this example, proteins isolated from two B cell lines, 9062 which reacted strongly to S1 and 9060, which was weakly reactive to S1, were selected for HLA peptidome analysis in order to identify the specific HLA Class I peptides that were presumably bound to the groove of DQβ0603:DQα0103 proteins and recognized by S1.

Multiple peptides located between amino acid position 119-148 of the mature HLA Class I heavy chain were identified by mass spectrometry analysis (Table 2). Interestingly, only peptides from this region were identified from the peptidome analysis regardless of whether the peptides were derived from HLA-A, -B, -C, -E or -F. Peptides that contain a polymorphic sequence at amino acid position 142 for HLA A*02:01 were present in the antigen prepared from 9062 and for A*01:01 in antigen prepared from 9060 suggesting the polymorphic A*02:01 peptide might be involved in the S1 antibody epitope.

### Example 5

### DQβ0603:DQα0103 Antibodies Recognize Different Amino Acid Sequences of Same but Polymorphic Class I Region

According to this example, one peptide with the same length between 9060 and 9062 from each Class I gene in the peptidome analysis was synthesized and used to verify the ability to restore the antibody reactivity after loading it onto HLA Class I negative recombinant DQβ0603:DQα0103 proteins in FIG. 2A in order to confirm the expected peptide fragments of the Class I proteins that participated in the DQβ0603:DQα0103 reactive epitope. Additionally, peptides with the sequences from the same location of other common alleles that were not presented in the peptidome analysis were also synthesized and tested. Biotin was incorporated at the N-terminal of the peptides to measure the efficiency of the peptide loading process (Table 3). Peptides were loaded onto the DQβ0603:DQα0103 protein from the bead panel in FIG. 2A and their peptide binding efficiencies were measured by the reactivity of R-Phycoerythrin Conjugated Streptavidin (SAPE) with biotin at the N-terminal of the peptides.

The DQβ0603:DQα0103 protein from BLS cell line showed the highest SAPE signals with all the peptides (data not shown) and its interaction with the DQβ0603:DQα0103 antibodies was examined. All six sera reacted to at least one peptide loaded protein (FIG. 4A-F). SAPE reacted to all HLA peptides with similar intensity confirming the binding of HLA peptides by sera S1-S6 were peptide-specific (FIG. 4G). Serum S1 reacted primarily with A2 peptide from A*02:01 and weakly with peptides from other HLA alleles including A1 peptide which sequence was present in HLA A locus alleles from weak S1 reactive B cell lines, 9060, 9065 and 9105 (FIG. 4A). There is only one amino acid difference at amino acid position 142 between A2 and A1 peptide that determined the specificity of the antibody in Serum S1. Serum S6 reacted to a core sequence “SWTAADT” shared by most of the HLA B and some C alleles and HLA-F and was completely negative with the three peptides in A locus (A1-A3) confirming the negative reactivity with proteins isolated from the T2DM and LCL3023 230 transfectants seen previously (FIG. 4F).

TABLE 3
Synthetic peptide sequence and the matching serological groups.

| ID | Sequence | HLA sero-group |
|---|---|---|
| A1* | Bio-EDLRSWTAADMAAQITKR | A1, A3, A11, A24, A36, A80 |
| A2* | Bio-EDLRSWTAADMAAQTTKH | A2, A68, A69 |
| A3 | Bio-EDLRSWTAADMAAQITQR | A23, A25, A26, A29, A30, A31, A32, A33, |
| | | A34, A43, A66, A74 |
| B1* | Bio-EDLSSWTAADTAAQITQRKWE | B14, B15, B18, B27, B35, B37, B38, B39, |
| | | B44, B45, B46, B47, B49, B50, B51, B52, |
| | | B53, B54, B55, B56, B57, B58, B59, B67, |
| | | B78, B82 |
| B2 | Bio-EDLSSWTAADTAAQITQLKWE | B13 |
| B3 | Bio-EDLRSWTAADTAAQISQRKLE | B60, B48, B81, C17 |
| C1* | Bio-EDLRSWTAADTAAQITQRKWE | B7, B8, B61, B41, B42, C1, C2, C3, C4, C6, |
| | | C7, C0801, C12, C14, C15, C16, C18 |
| C2 | Bio-EDLRSWTAADKAAQITQRKWE | C5, C0802 |
| E1* | Bio-EDLRSWTAVDTAAQISE | E |
| F1* | Bio-EDLRSWTAADTVAQITQ | F |
| DRA1* | Bio-KPVTTGVSETVFLPREDH | DRA |

Peptides with * were derived from the peptidome analysis in Table 2

Serum S5 reacted to all three A locus sequences suggesting that the recognition site might be centered on the M (Methionine) at amino acid position 138 and the nearby sequence but does not extend past Q (Glutamine) at amino acid position 141 (FIG. 4E). Assuming all these peptides were binding to the groove of DQβ0603:DQα0103 through the same anchor positions, it suggested that the amino acid sequences of DQβ0603:DQα0103 participating in the antibody epitopes were not the same for all the DQβ0603:DQα0103 antibodies. Serum S4 reacted to all Class I peptides except C2 and E with the likely recognized sequence of 136ADT/M138 (FIG. 4D). The positive charge of 138K on C2 was most likely the reason for the negative reaction of S4. Serum S2 reacted to most of the peptides with various degrees of intensity (FIG. 4B). Serum S3 has similar but weaker reactivity than S2 (FIG. 4C).

The different reactivity observed for peptides from amino acid position 128-145 of the polymorphic region on Class I heavy chain loaded in DQβ0603:DQα0103 protein supports the conclusion that DQβ0603:DQα0103 antibodies can differentiate peptides with polymorphic sequences from different loci and alleles.

### REFERENCES

1. Patel, R. & Terasaki, P. I. Significance of the Positive Crossmatch Test in Kidney Transplantation. N Engl J Med. 280, 735-739 (1969).
2. Tambur, A. R. & Das, R. Can we use eplets (or molecular) mismatch load analysis to improve organ allocation? The hope and the hype. Transplantation 107, 605-615 (2023).
3. Fuller, T. C., Fuller, A. A., Golden, M. & Rodey, G. E. HLA Alloantibodies and the 468 Mechanism of the Antiglobulin-Augmented Lymphocytotoxicity Procedure. Hum Immunol. 56, 94-105 (1997).
4. Garovoy, M, R. et al. Flow cytometry analysis: a high technology cross-match technique 471 facilitating transplantation. Transplant Proc. 15, 1939-1944 (1983). 472
5. Pei, R., Lee, J.-H., Shih, N.-J., Chen, M. & Terasaki, P. I. Single human leukocyte antigen 473 flow cytometry beads for accurate identification of human leukocyte antigen antibody 474 specificities. Transplantation. 75, 43-49 (2003).
6. Friedewald, J. J. et al. The kidney allocation system. Surg. Clin. North Am. 93, 1395-476 1406 (2013).
7. Bray, R. A. The acceptability and application of virtual crossmatching in lieu of serologic 478 crossmatching for transplantation. Virtual Crossmatch Work Group Report. Centers for 479 Disease Control and Prevention. https://www.cdc.gov/cliac/docs/addenda/cliac1114/
8. BRAY_Virtual_Cross 481 match_Workgroup_Report_Nov-2014.pdf (2014). 482 8. Wehmeier, C., Honger, G. & Schaub, S. Caveats of HLA antibody detection by solid phase assays. Transpl. Int. 33, 18-29 (2020).
9. Grenzi, P. C., de Marco, R., Silva, R. Z., Campos, E. F. & Gerbase-DeLima, M. Antibodies against denatured HLA class II molecules detected in luminex-single antigen assay. Hum. Immunol. 74, 1300-1303 (2013).
10. Dean, C. L., Krummey, S. M., Gebel, H. M., Bray, R. A. & Sullivan, H. C. Identification of a recurrent pattern of false-positivity by Luminex HLA MHC class I single antigen bead testing. Hum. Immunol. 81, 73-78 (2020).
11. Frischknecht, L. et al. The impact of pre-transplant donor specific antibodies on the outcome of kidney transplantation-Data from the Swiss transplant cohort study. Front. Immunol. 13:1005790. doi: 10.3389/fimmu.2022.1005790. eCollection (2022).
12. Tambur, A. R. et al. Significance of HLA-DQ in kidney transplantation: time to reevaluate human leukocyte antigen-matching priorities to improve transplant outcomes? An expert review and recommendations. Kidney Int. 100, 1012-1022 (2021).
13. Gorga, J. C., Horejsi, V., Johnson, D. R., Raghupathy, R. & Strominger, J. L. Purification 497 and characterization of class II histocompatibility antigens from a homozygous human B 498 cell line. J. Biol. Chem. 262, 16087-16094 (1987).
14. Monos, D. S. et al. L cells expressing DQ molecules of the DR3 and DR4 haplotypes: 500 reactivity with mAbs. Immunogenetics 42, 172-180 (1995).
15. Denzin, L. K. & Cresswell, P. HLA-DM induces CLIP dissociation from MHC class II 502 alpha beta dimers and facilitates peptide loading. Cell 82, 155-165 (1995).
16. Ceman, S., Rudersdorf, R., Long, E. O. & Demars, R. MHC class II deletion mutant 504 expresses normal levels of transgene encoded class II molecules that have abnormal 505 conformation and impaired antigen presentation ability. J. Immunol. 149, 754-761 (1992).
17. Ceman, S., Rudersdorf, R. A., Petersen, J. M. & DeMars, R. DMA and DMB are the only 507 genes in the class II region of the human MHC needed for class II-associated antigen 508 processing. J. Immnol. 154, 2545-2556 (1995).
18. Tait, B. D. et al. Consensus guidelines on the testing and clinical management issues 510 associated with HLA and non-HLA antibodies in transplantation. Transplantation 95, 19-511 47 (2013).
19. Menezes Teles E Oliveira, D. et al. pHLA3D: Updating the database of predicted three513 dimensional structures of HLA with HLA-DR, HLA-DQ and HLA-DP molecules. Hum. 514 Immunol. 82, 8-10 (2021).
20. Kim, B., Kim, S., Park, Y. & Kim, H.-S. False-positive reactivity of anti-human leukocyte 516 antigen antibodies detected using the single-antigen bead assay. Hum. Immunol. 82, 409-517 413 (2021).
21. Reid, S. W. et al. Antagonist HIV-1 Gag Peptides Induce Structural Changes in HLA B8. 519 J. Exp. Med. 184, 2279-2286 (1996).
22. Halloran, P. F. et al. The Trifecta Study: Comparing Plasma Levels of Donor-derived 521 Cell-Free DNA with the Molecular Phenotype of Kidney Transplant Biopsies. J. Am. Soc. 522 Nephrol. 33, 387-400 (2022).
23. Halloran, P. F. et al. Antibody-mediated Rejection Without Detectable Donor-specific Antibody Releases Donor-derived Cell-free DNA: Results From the Trifecta Study. Transplantation 107, 709-719 (2023).
24. Stern, L. J. & Wiley, D. C. Antigenic peptide binding by class I and class II 527 histocompatibility proteins. Structure 2, 245-251 (1994).
25. Salter, R. D., Howell, D. N. & Cresswell, P. Genes regulating HLA class I antigen 529 expression in T-B lymphoblast hybrids. Immunogenetics. 21, 235-246 (1985).
26. Burlingham, W. J., Ceman, S. S. & Demars, R. Secretion and cell surface expression of 531 IgG1 are impaired in human B lymphoblasts that lack HLA-A, -B, and -C antigens. Proc. 532 Natl. Acad. Sci. USA 86, 8005-8009 (1989).

What is claimed is:

1. A method of determining whether antibody reactivity against an HLA Class 2 antigen is dependent upon the presence of a peptide bound to the peptide binding groove of said HLA Class 2 antigen comprising the steps of:
creating a panel comprising at least two of the same HLA Class 2 antigens with different peptides bound to the peptide binding grooves of thereof,
contacting a sample containing antibodies specific for at least one HLA Class 2 antigen and
determining whether an antibody which binds to a first HLA Class 2 antigen with a first peptide bound to the peptide binding groove thereof binds to said first HLA Class 2 antigen with a second peptide bound to the peptide binding groove thereof.

2. The method of claim 1 wherein the peptide is an HLA Class 1 peptide.

3. The method of claim 1 wherein the peptide is derived from amino acids 119-148 of the HLA Class 1 heavy chain.

4. The method of claim 1 wherein the HLA Class 2 antigen is a DQ antigen.

5. The method of claim 1 wherein the HLA Class 2 antigen is a DQβ0603:DQα0103 antigen.

6. Solid substrates having an HLA Class 2 antigen bound thereto wherein the HLA Class 2 antigen has a peptide bound to the peptide binding groove thereof.

7. The solid substrate of claim 6 wherein the peptide is an HLA Class 1 peptide.

8. The solid substrate of claim 6 which comprises a single antigen bead (SAB).

9. An assay device comprising a solid phase array of at least two of the same HLA Class 2 antigens with different peptides bound to the peptide binding grooves thereof.

10. The assay device of claim 9 wherein the different peptides are HLA Class 1 peptides.

11. The assay device of claim 9 wherein the solid phase array comprises multiple assay beads wherein at least two beads present of the same HLA Class 2 antigens with different HLA Class 1 peptides bound to the peptide binding groove thereof.

12. An assay device comprising a solid phase array of at least two of the same HLA Class 2 antigens wherein a first HLA Class 2 antigen has a peptide bound to the peptide binding groove thereof and a second HLA Class 2 antigen has no peptide bound to the peptide binding groove thereof.

13. The assay device of claim 12 wherein the peptide bound to a first HLA Class 2 antigen is an HLA Class 1 antigen.

14. A method of tissue typing for determining transplant compatibility comprising contacting a serum sample from a potential transplant recipient to a solid substrate having an HLA Class 2 antigen bound to the peptide binding groove thereof wherein the HLA Class 2 antigen has a peptide bound to the peptide binding groove thereof.

15. The method of claim 14 wherein the peptide bound to the HLA Class 2 antigen is an HLA Class 1 peptide.

16. The method of claim 14 wherein the HLA Class 1 peptide is derived from amino acids 119-148 of the HLA Class 1 heavy chain.

17. The method of claim 14 wherein the HLA Class 2 antigen is a DQ antigen.

18. The method of claim 14 wherein the HLA Class 2 antigen is an DQβ0603:DQα0103 antigen.

19. A method of detecting DQβ0603:DQα0103 specific antibodies comprising contacting a sample suspected of containing said antibodies with an HLA Class 2 antigen having an HLA Class 1 peptide bound to the peptide binding groove thereof.

20. The method of claim 19 wherein the sample is contacted with at least two of the same HLA Class 2 antigens wherein a first HLA Class 2 antigen has an HLA Class 1 peptide bound to the peptide binding groove thereof and a second HLA Class 2 antigen has a different HLA Class 1 peptide bound to the peptide binding groove thereof.
